(19) United States
(12) Patent Application Publication
(10) Pub. No.: US 2022/0202968 A1
(43) Pub. Date: Jun. 30, 2022

(54) FAR-UVC GERMICIDAL SYSTEM

(71) Applicant: LED Smart Inc., Surrey (CA)

(72) Inventor: Xinxin Shan, Surrey (CA)

(73) Assignee: LED Smart Inc., Surrey (CA)

(21) Appl. No.: 17/175,526

(22) Filed: Feb. 12, 2021

(30) Foreign Application Priority Data
Dec. 25, 2020 (CA) 3,104,168
Jan. 15, 2021 (CA) 3,105,865

(51) Int. Cl. A61L 2/00; A61L 2/10; A61L 2/26

(52) U.S. Cl. CPC A61L 2/0047; A61L 2/10; A61L 2/26; A61L 2202/26; A61L 2202/121; A61L 2202/14; A61L 2202/11

(57) ABSTRACT

A germicidal lighting system uses far-UVC lamps emitting frequencies that do not harm humans, for example 222 nm. The presence of a person is detected using sensors, and the far-UVC lamps are turned on when a person is present and off when no one is present. This may be applied to a person's whole body or, for example, a hand or hands. A far-UVC optical system for a germicidal lighting system may include a wavelength selective mirror arranged to receive far-UVC light from a far-UVC light source and to disproportionately reflect the far-UVC light in at least one wavelength of the far-UVC light relative to at least another wavelength of light emitted by the far-UVC light source. A far-UVC light source may be placed in a reflective enclosure to direct light through an opening. The reflective enclosure or a mirror to which the light is directed may be rotated.

### TECHNICAL FIELD

Germicidal lighting systems.

### BACKGROUND

UVC light is used as a disinfection tool. For example, 254 nm UVC light from mercury lamps has been commonly used. However, many wavelengths of UVC including 254 nm cause radiation damage to the skin of human and animals. A range of wavelengths have been found to be much less damaging to humans and animals. This range, referred to in this document as “far-UVC”, has a smaller distance of penetration through biological material and in particular cannot penetrate either the human stratum corneum (the outer dead-cell skin layer), nor the ocular tear layer, nor even the cytoplasm of individual human cells. However, it can still penetrate bacteria and viruses which are much smaller than human cells. Examples of wavelengths within this range include 207 nm, as produced by a Kr—Br excimer lamp, and 222 nm, as produced by a Kr—Cl excimer lamp. These are peak wavelengths provided by these excimer lamps. Each excimer lamp may produce a range of wavelengths, and studies have used filters to exclude light beyond, e.g., a nanometer or so from the respective peaks, or outside a range believed to be non-harmful to humans. Studies have focused on these specific excimer lamp wavelengths, but the reasons for their non-harmfulness to humans and effectiveness on bacteria and viruses can be expected to apply to a range of wavelengths that will extend some distance shorter than 207 nm, longer than 222 nm, and everything in between. The term “far-UVC” is used in this document to refer to 207 nm, 222 nm, and the full contiguous range of wavelengths, including 207 and 222 nm and extending to shorter wavelengths than 207 nm, longer wavelengths than 222 nm, and intermediate wavelengths, that is germicidally effective while being substantially nonharmful to humans. For example, the range of 200 nm to 230 nm may be suitable. In another example, proposed by U.S. Pat. No. 10,786,586, the suitable range may be 190 nm to 237 nm.

### SUMMARY

There is provided a far-UVC optical system for a germicidal lighting system. The far-UVC optical system includes a far-UVC light source and a wavelength selective mirror arranged to receive far-UVC light from the far-UVC light source and to disproportionately reflect the far-UVC light in at least one wavelength of the far-UVC light relative to at least another wavelength of light emitted by the far-UVC light source.

In various embodiments, there may be included any one or more of the following features: the wavelength selective mirror may have a wavelength selective coating over a base surface of the wavelength selective mirror. The wavelength selective coating may include a chemical that absorbs the at least another wavelength and does not substantially absorb the at least one wavelength. The base surface may be a reflective surface. The wavelength selective coating may include one or more dielectric layers of thickness and refractive index selected to reflect the at least one wavelength. The far-UVC optical system may have plural mirrors arranged in a sequence where each successive mirror of the sequence receives light reflected from a corresponding previous mirror of the sequence, the wavelength selective mirror being a mirror of the plural mirrors arranged in the sequence. The wavelength selective mirror may be one of plural wavelength selective mirrors in the sequence of mirrors. A blocking member may be arranged to block light from the far-UVC light source that would, if not blocked, avoid the wavelength selective mirror, or if there is a sequence of mirrors, the first mirror of the sequence. The blocking member may be a further mirror arranged to reflect light from the far-UVC light source to the wavelength selective mirror, or if there is a sequence of mirrors, the first mirror of the sequence. A light diffusion board may be arranged to receive the far-UVC light from the wavelength selective mirror, or if there is a sequence of mirrors, from the sequence of mirrors, and to distribute the light to form an output of the far-UVC optical system. The wavelength selective mirror may be concave, flat or convex. The far-UVC light source may include an excimer lamp.

There is provided a germicidal lighting system for disinfecting skin or clothing of a human. The germicidal lighting system includes a structure defining an opening for accommodating the human or a body part of the human. One or more sensors are arranged on the structure to generate sensor information indicative of the presence of the human or body part within the opening. One or more far-UVC lamps are arranged on the structure to emit far-UVC light to disinfect the human or human body part when the human or human body part is positioned within the opening. A processor is connected to the sensors and to the far-UVC lamps and configured to analyze the sensor information to determine that the human or body part is present within the opening, and to activate the far-UVC lamps based on the determination that the human or body part is present within the opening.

In various embodiments, there may be included any one or more of the following features: light emitted by the one or more far-UVC lamps has a wavelength within the range of 207 to 222 nm. The wavelength may be 222 nanometers. The processor may be configured to analyze the sensor information to determine that the human or human body part is no longer present in the opening, and to deactivate the one or more far-UVC lamps based on the determination that the human or body part is no longer present within the opening. The opening may be a receptacle arranged to receive a hand or hands. The hand or hands may be two hands, and the processor may be configured to activate the one or more far-UVC lamps based on the determination that both hands are present simultaneously. The germicidal lighting system may include a signal generation system, and the processor may be configured to cause the signal generation system to generate a finishing signal based on the far-UVC light having been active for an amount of time sufficient to disinfect the hand or hands. The processor may be configured to determine from the sensor information whether the hand or hands are remaining in the opening, and to cause the signal generation system to send the finishing signal based on the hand or hands having been within the opening for the amount of time sufficient to disinfect. The processor may be configured to cause the warning signal generation system to generate a warning signal based on the hand or hands not remaining within the opening. The sensor information may also be indicative of a position of the hand or hands in the opening, and the processor is configured to analyze the sensor information to determine whether the hand or hands are in a pre-selected position, and to cause the signal generation system to send the warning signal based on the hand or hands not being in the pre-selected position and to send the finishing signal based on the hand or hands having been in the pre-selected position for the amount of time. The sensor information may also be indicative of a position of the hand or hands in the opening, and the processor may be configured to analyze the sensor information to determine whether the hand or hands are in a pre-selected position, and to cause the signal generation system to send the finishing signal based on the hand or hands having been in the pre-selected position for the amount of time. The opening may be a pedestrian passage. The structure may comprise a gateway defining the pedestrian passage. The structure may comprise a moving walkway defining the pedestrian passage. The one or more far-UVC lamps are plural far-UVC lamps, each far-UVC lamp of the plural far-UVC lamps corresponding to a respective illumination area within the pedestrian passage, the one or more sensors being arranged to generate sensor information indicative of a position of a human along the pedestrian passage, and the processor may be configured to analyze the sensor information to associate the human with an illumination area and to activate a far UVC lamp of the plural far-UVC lamps based on the correspondence between the far-UVC lamp of the plural far-UVC lamps and the illumination area to which the human is associated. The far-UVC lamps may each comprise a concave mirror and corresponding light source, the mirror arranged to reflect the far-UVC light from the corresponding light source into collimated light output. The mirror and corresponding light source may be adjustable in distance from each other to produce diverging or converging light output. The processor may be multiple processors. The one or more sensors may include a far-UVC intensity sensor, the processor being configured to determine an intensity of the far-UVC light based on information from the far-UVC intensity sensor, and to cause the generation of a warning signal indicating that the one or more far-UVC lamps need to be replaced based on the determined intensity of the far-UVC light. The one or more sensors may include a body sensor, the processor being configured to determine a size of a human body present at the opening based on information from the body sensor, and to operate the one or more far-UVC lamps in part based on the determined size. The one or more far-UVC lamps may include a far-UVC optical system as described above.

There is provided a far-UVC optical system for a germicidal lighting system, the far-UVC optical system having a far-UVC light source and a reflective enclosure around the far-UVC light source, the reflective enclosure having an opening and having reflective walls arranged to direct far-UVC light from the far-UVC light source to the opening.

In various embodiments, there may be included any one or more of the following features: the reflective walls may include at least one concave portion. The opening may be out of direct line of sight from at least a center of the far-UVC light source. The reflective walls may be arranged as a spiral. Alternatively, the reflective walls may include portions arranged around a direct line of sight from the far-UVC lamp to an intermediate position in the optical system to reflect generally to the intermediate position light emitted from the far-UVC lamp in directions other than the direct line of sight from the far-UVC lamp to the intermediate position. These portions arranged around the direct line of sight may be concave portions. The far-UVC light source may be a linear light source. The concave portions may be shaped in cross section to form portions of one or more ovals in a plane perpendicular to the light source. The concave portions may be shaped in cross section to form portions of one or more conic sections in a plane perpendicular to the light source. The reflective walls also include an end mirror at the intermediate position in the optical system, the end mirror arranged to direct to the opening the far-UVC light from the concave portions of the reflective walls and from the direct line of sight from the far-UVC lamp. There may also be a second end mirror in a second direct line of sight from the far-UVC lamp and arranged to direct the far-UVC light through a second opening, the second end mirror located at a second intermediate position of the optical system and second concave portions being arranged around the second direct line of sight from the far-UVC lamp to guide generally to the second intermediate position light emitted from the far-UVC lamp in directions other than the second direct line of sight from the far-UVC lamp to the second intermediate position. The second end mirror is arranged opposite to the end mirror.

The reflective walls may include a wavelength selective mirror arranged to receive the far-UVC light from the far-UVC light source and to disproportionately reflect the far-UVC light in at least one wavelength of the far-UVC light relative to at least another wavelength of light emitted by the far-UVC light source. The reflective enclosure may be movable. The reflective enclosure may be rotatable. The reflective enclosure may be rotatable about an axis and the reflective enclosure may be configured to output the far-UVC light from the opening so that the light spreads further in a direction parallel to the axis than in a direction perpendicular to the axis. The far-UVC light source may be a linear light source with a direction of linear extent parallel to the axis. The far-UVC optical system may also include an actuator connected to rotate the reflective enclosure. The actuator may be a stepper motor. The actuator may be configured to rotate the reflective enclosure at a variable speed, the speed depending on the distance from the opening to at least a portion of a target to be irradiated by the far-UVC optical system.

The far-UVC optical system including a reflective enclosure may also include an additional mirror arranged to receive the far-UVC light after it exits the opening. The additional mirror may be a wavelength selective mirror. The additional mirror may be rotatable. The additional mirror may be rotatable about an axis and the reflective enclosure may be configured to output the far-UVC light from the opening so that the light spreads further in a direction parallel to the axis than in a direction perpendicular to the axis. the far-UVC light source may be a linear light source with a direction of linear extent parallel to the axis. There may be an actuator connected to rotate the additional mirror. The actuator may be a stepper motor. The actuator may be configured to rotate the additional mirror at a variable speed, the speed depending on the distance from the additional mirror to at least a portion of a target to be irradiated by the far-UVC optical system.

The far-UVC optical system including a reflective enclosure may also include a sequence of additional mirrors where each successive mirror of the sequence receives light reflected from a corresponding previous mirror of the sequence, the first mirror of the sequence receiving light from the opening of the reflective enclosure. The reflective enclosure may be wavelength selective and/or at least one of the mirrors of the sequence of mirrors may be a wavelength selective mirror. The sequence of mirrors may include a rotatable mirror. The rotatable mirror may be rotatable about an axis and the reflective enclosure is configured to output the far-UVC light from the opening so that the light spreads further in a direction parallel to the axis than in a direction perpendicular to the axis. The far-UVC light source may be a linear light source with a direction of linear extent parallel to the axis. There may be an actuator connected to rotate the rotatable mirror. The actuator may be a stepper motor. The actuator may be configured to rotate the rotatable mirror at a variable speed, the speed depending on the distance from the rotatable mirror to at least a portion of a target to be irradiated by the far-UVC optical system.

These and other aspects of the device and method are set out in the claims.

### BRIEF DESCRIPTION OF THE FIGURES

Embodiments will now be described with reference to the figures, in which like reference characters denote like elements, by way of example, and in which:

FIG. 1 is an isometric view of an example of a far-UVC germicidal system suitable for sanitizing hands;

FIG. 2 is an isometric view of another example of a far-UVC germicidal system suitable for sanitizing hands;

FIG. 3 is a schematic cross section view of a far-UVC germicidal system suitable for sanitizing hands;

FIG. 4 is a flow chart showing an example method of operation of a far-UVC germicidal lighting system suitable for sanitizing hands;

FIG. 5 is an isometric view of a far-UVC germicidal system arranged as a gateway for disinfecting skin or clothing of people who pass through the gateway;

FIG. 6 is a closeup isometric view of an end of a moving sidewalk arranged as a far-UVC germicidal system for disinfecting people who move along the moving sidewalk;

FIG. 7 is an isometric view of the moving sidewalk shown in closeup in FIG. 6;

FIG. 8 is a schematic cross-section view of a far-UVC optical system using a wavelength selective mirror;

FIG. 9 is a schematic cross section view of another far-UVC optical system using a wavelength selective mirror;

FIG. 10 is a schematic cross section view of a far-UVC optical system using a wavelength selective mirror as part of a sequence of mirrors;

FIG. 11 is a schematic cross section view of a far UVC optical system using a light diffusion board;

FIG. 12 is a schematic cross section view of an example wavelength selective mirror using dielectric layers;

FIG. 13 is a schematic cross section view of another example of a wavelength selective mirror using dielectric layers;

FIG. 14 is a schematic cross section view of a far-UVC optical system using a reflective enclosure around a far-UVC light source;

FIG. 15 is a schematic cross section view of a far-UVC optical system using a reflective enclosure around a far-UVC light source, and an additional mirror;

FIG. 16 is a schematic cross section view of a far-UVC optical system using a reflective enclosure around a far-UVC light source, and a sequence of additional mirrors; and

FIG. 17 is an end view of a far-UVC optical system using a reflective enclosure showing a motor for rotating the reflective enclosure; and

FIG. 18 is a cross-section view of a far-UVC optical system using an enclosure with two openings.

### DETAILED DESCRIPTION

Immaterial modifications may be made to the embodiments described here without departing from what is covered by the claims.

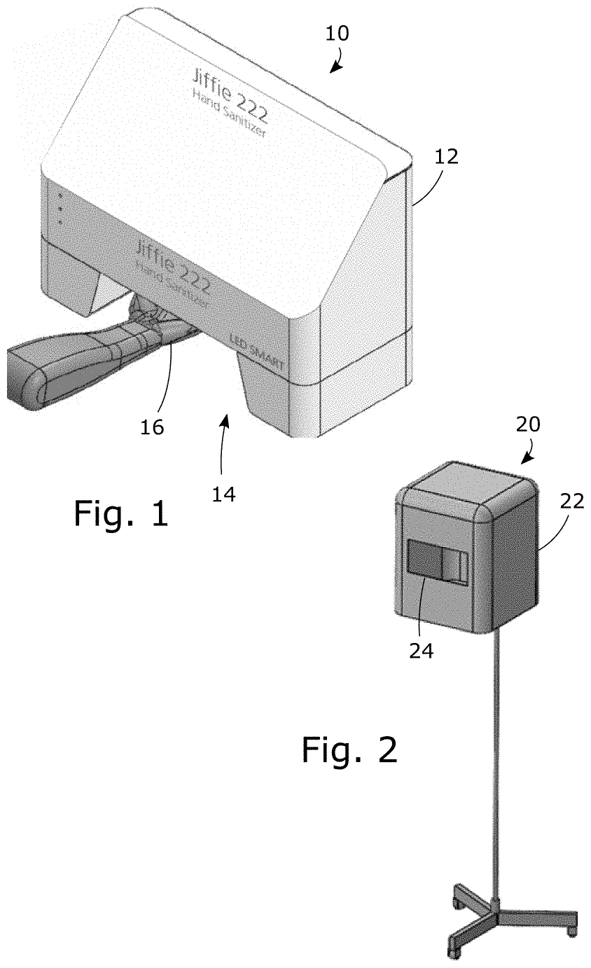

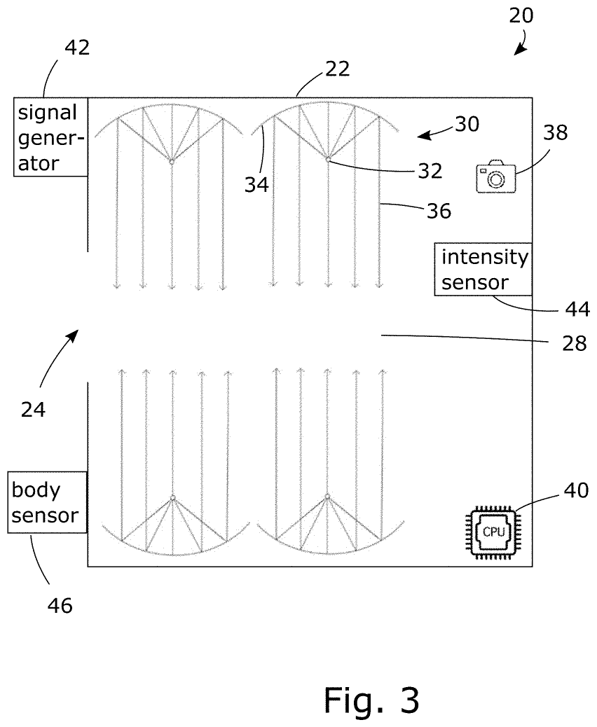

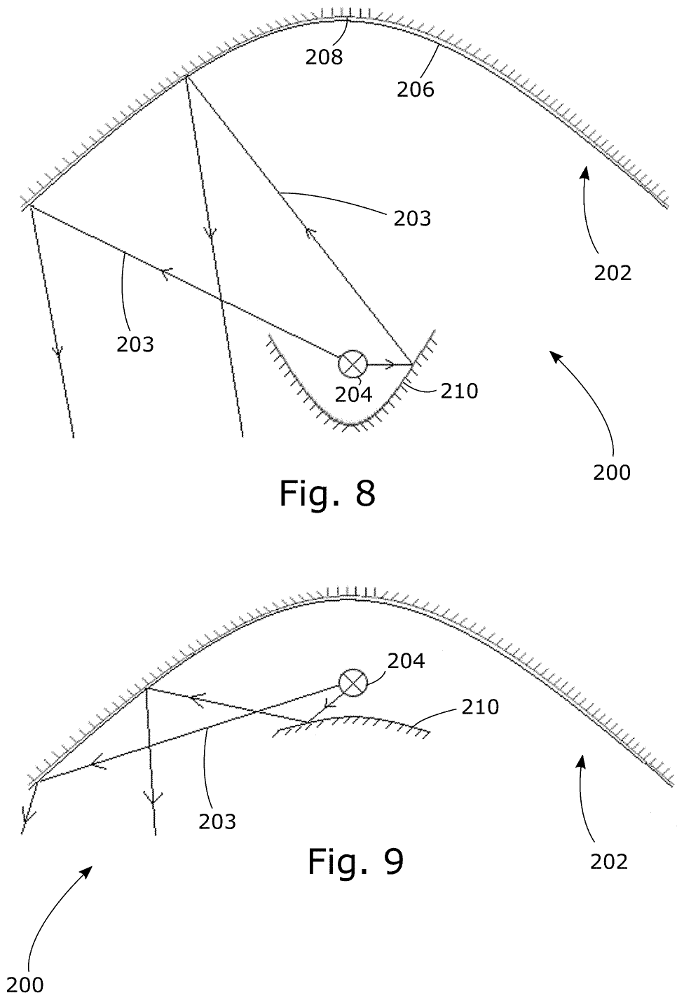

A far-UVC germicidal lighting system, for example using 222 nm far-UVC light, may be used to disinfect the skin or worn clothing of a human due to its non-harmfulness to humans. 222 nm light may be produced for example by a filtered Kr—Cl excimer lamp. A far-UVC germicidal lighting system may comprise a structure defining an opening for accommodating a human or a body part of a human. As shown in a first example in FIG. 1, far-UVC germicidal lighting system 10 may comprise a structure 12 defining an opening 14, here an opening arranged to accommodate a human hand or hands 16. In the example shown in FIG. 1, the opening is defined in part by the structure 12 and in part by a surface (not shown) on which the structure may be placed. In an alternate example of a hand sanitizing germicidal lighting system 20 shown in FIG. 2, an opening 24 for a human hand or hands may be defined wholly by a structure 22 of the germicidal lighting system 20. FIG. 3 shows a schematic section view of a hand sanitizing germicidal lighting system 20 as in FIG. 2. Opening 24 leads into internal cavity 28. At the top and bottom of internal cavity 28 are far-UVC lamps 30. In the example shown, each far-UVC lamp 30 includes a far-UVC light source 32 and a concave mirror 34 here arranged to direct far-UVC light into parallel, convergent, or divergent rays 36, here parallel (collimated) light. Each far-UVC lamp may include a filter (not shown in FIG. 3) for excluding light outside the desired passband of far-UVC light (for example, 200 nm to 230 nm, 190 nm to 230 nm, 190 nm to 237 nm or other passband). The filter may be a conventional absorption filter placed at an exit of the UVC lamp, or a wavelength selective mirror as further described below in relation to FIGS. 8-11. The distance between the light source and mirror for each light may also be adjustable. In an example, reducing the distance between the light source and mirror leads to diverging light and increasing the distance leads to converging light.

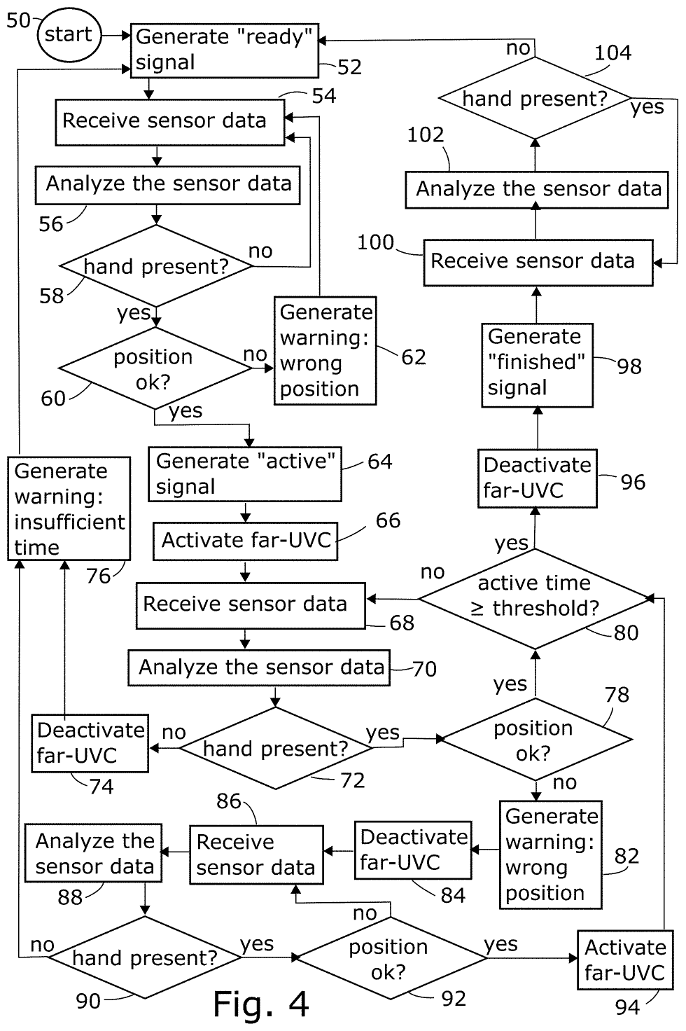

One or more sensors 38 are arranged to generate sensor information indicative of the presence of a hand or hands in the cavity. The one or more sensors 38 are connected to send the sensor information to a processor 40 which is configured to analyze the sensor information to determine whether the hand or hands are present within the opening. The processor 40 is depicted as within structure 22 but may be located elsewhere and connected remotely. The processor may comprise multiple processors, and the multiple processors may be located at different locations. The one or more sensors 38 are schematically represented as an image sensor but may include a variety of sensors. The processor 40 is connected to the far-UVC lamps 30 to control the far-UVC lamps based on the analysis of the sensor information, for example as shown in FIG. 4. A signal generation system represented by signal generator 42 may be connected to the processor and instructed by the processor to produce signals to inform the user of the status of the disinfection procedure, as described in more detail in relation to FIG. 4 below. The signal generation system 42 may generate, for example, visual and/or audio signals. The signals generated can include progress indication signals including, for example, a finishing signal, and warning signals indicating problems. Each signal may comprise a single or multiple cues directed to a single or multiple sensory modality (e.g. audio, visual). The signal generation system 42 may comprise a single signal generator or multiple signal generators. Multiple signal generators may generate, for example, cues directed to different sensory modalities (e.g. audio or visual), different signals or cues within a modality, or simply provide additional cues for e.g. redundancy. Where there are multiple signal generators, they may be located separately or together. The one or more sensors 38 may also include a far-UVC intensity sensor 44 to monitor the intensity of the far-UVC radiation. A far-UVC germicidal lighting system processor 40, for example using a process as described below in relation to FIG. 4, may use the intensity sensor 44 to determine if the one or more far-UVC lamps 30 need to be replaced, for example due to low intensity of far-UVC light. In that event, the processor 40 may be configured to cause the signal generator 42 to generate a signal indicating that the one or more far-UVC lamps 30 need to be replaced. Optionally, the processor may halt the operation of the germicidal lighting system until the replacement occurs and, e.g., the system is reset. A body sensor 46 may also be included to determine if a human body is present, and may also detect the approximate size. The body sensor may be used to prevent misuse by children, for example by the processor 40 stopping the germicidal lighting system if no human body is detected or the detected human body is too small. The body sensor 46 could also prevent repeated misuse by the processor determining using the body sensor 46 if a single person has remained present between multiple uses of the system, and to halt the system in this event.

FIG. 4 is a flow chart illustrating an example process by which a processor may control a far-UVC lighting system such as a hand sanitizer as illustrated in FIGS. 1-3. As noted above, the processor may be multiple processors, and the multiple processors need not be co-located. When turned on, the processor proceeds from start 50 to cause a signal generation system to generate a ready signal in step 52 and receive sensor data in step 54. The processor then analyzes the sensor data in step 56. The analysis may include, for example, a comparison of the sensor data to preset criteria, or machine learning based processing, or a combination of different analysis techniques. From the analysis, the processor may for example, in the case of a hand sanitizer, determine whether a hand is present in the opening of the hand sanitizer and whether the hand is in one of possibly several pre-selected positions. In decision step 58, if no hand is present, the processor returns to step 54 to receive new sensor data at a new time step. If a hand is present, the processor proceeds to step 60. In decision step 60, if the hand is not appropriately positioned, i.e. not in a pre-selected position, the processor causes the signal generation system to generate a “wrong position” warning signal in step 62, and returns to step 54 to receive new sensor data at a new time step. If the hand is appropriately positioned, the processor proceeds to generate an “active” signal in step 64 and activate the far-UVC lamp(s) in step 66. In step 68, the processor continues to receive further sensor data, and analyzes the further sensor data in step 70. The sensing and analysis in steps 68 and 70 may be the same as or different from the sensing and analysis in steps 54 and 56. Again, the analyses may include determining whether a hand is present and whether it is appropriately positioned. In decision step 72, if the hand has been removed from the opening, the processor deactivates the far-UVC lamp(s) in step 74, and generates a warning signal 76 indicating that the hand was removed prematurely. The processor can then return to step 52 to generate a “ready” signal. If in decision step 72 the hand remains within the opening, the processor proceeds to decision step 78. In decision step 78, if the hands remain in the proper position, the processor proceeds to decision step 80. If the hands are no longer in the proper position, the analysis causes the signal generation system to generate a warning in step 82. This warning signal may be the same or different than the warning generated in step 62 where the far-UVC lamp(s) had not yet started. Along with generating the warning in step 82, the processor deactivates the far-UVC lamp(s) in step 84. The processor then receives sensor data in step 86, and analyzes the sensor data in step 88. The data and analysis may be the same as or different from that of steps 68 and 70 and steps 54 and 56. The processor then goes to decision step 90. In decision step 90, in the event that the hand has left the opening the processor then causes the signal generation system to generate a warning of insufficient time in step 76. If the hand remains present, the processor then goes to decision step 92. In decision step 92, if the hand is no longer in an appropriate position, the processor returns to receive sensor data in step 86. If the hand remains in an appropriate position, the processor reactivates the far-UVC lamp(s) in step 94, and proceeds to decision step 80. In decision step 80, the processor compares an active time of the far-UVC lamp(s) to a threshold. The active time may take into account, for example, the total time the far-UVC lamp(s) have been on since the hand entered the opening. Alternatively, for example, the active time may be reset whenever the hand is in an incorrect position. The time threshold may be a preset threshold or dynamically calculated based on, e.g. the amount and consistency of the dose received on the hand. The dose may be selected kill, for example, 99% of viruses on the hand, 99.9% of viruses on the hand, 99.99% of viruses on the hand, etc. The time threshold may also take into account the measured intensity of the far-UVC light, where the system includes a far-UVC intensity sensor 44. In decision step 80, if the time the far-UVC lamp(s) have been active is not larger than or equal to the threshold, the processor returns to receive sensor data for a further time step in step 68. If the time the far-UVC lamp(s) have been active has been larger than or equal to the threshold, then the processor proceeds to deactivate the far-UVC lamp(s) in step 96 and to cause the signal generation system to generate a “finished” signal in step 98. Alternatively, the far-UVC lamp(s) may be left on until the hand is removed, or until a further threshold is reached, to allow the choice of extra disinfection. The processor then proceeds to detect when the hand is removed in steps 100-104. The processor receives sensor data in step 100, and analyzes the sensor data in step 102. The data and analysis may be the same as or different from the data and analysis in steps 54 and 56, steps 68 and 70, and steps 86 and 88. In decision step 104, if the hand remains present, the processor returns to step 100 to receive more data. Once the hand has left the opening, the processor returns to step 52 to generate a “ready” signal.

In the method illustrated in FIG. 4, if the hand is in the wrong position or removed, the processor stops the far-UVC, but alternatively it could continue the far-UVC. It also pauses the activation time counter, but alternatively could continue the activation time counter or restart it. If the hand is removed the processor in the illustrated method stops the far-UVC and restarts the activation time counter, but it could alternatively continue the far-UVC and could continue or pause the activation time counter.

While the method shown in FIG. 4 is described and shown for one hand, it could also apply to two hands. For example, the processor could also require two hands to be present in the proper positions to start the far-UVC and the activation timer, and may pause or discontinue either or both of the far-UVC and the timer based on either or both hands no longer being present in the proper positions.

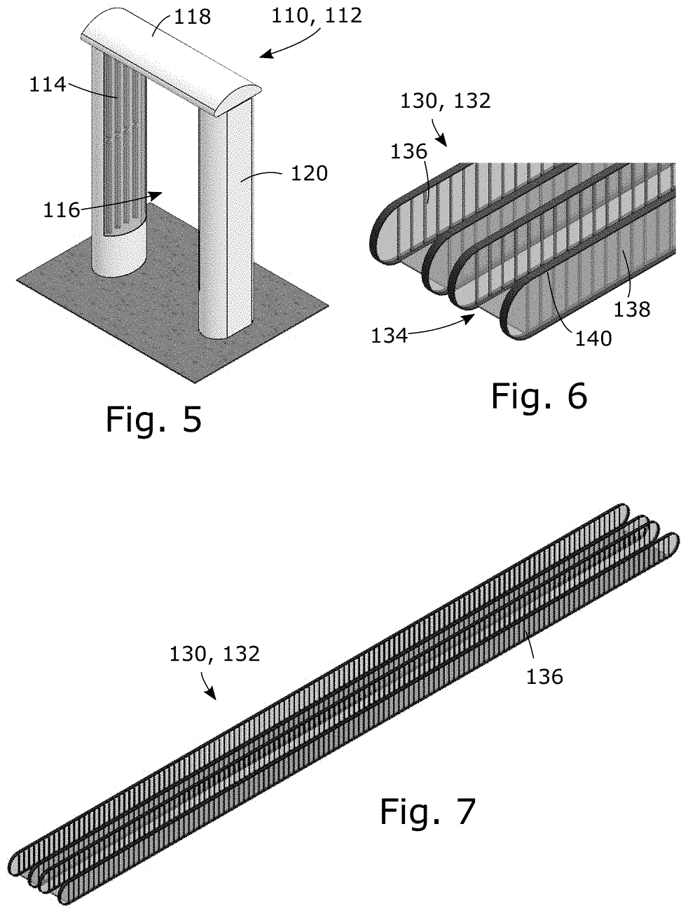

In the example embodiment shown in FIG. 1, the opening is defined in part by the structure 12 and in part by a surface (not shown) on which the structure may be placed. A hand placed into the opening may be against the surface on which the structure is placed, which may not have far-UVC lamps. As a result, far-UVC may not be applied to the portion of the hand against the surface. In order to fully disinfect the hand, the method shown in FIG. 4 may be modified to include instructions to change the orientation of the hand. For example, when checking the position of the hand in step 60 there may be plural pre-selected positions, at least one corresponding to each of two orientations. The processor may check to see if the hand is in a pre-selected position corresponding to a first orientation of the two orientations, or in a pre-selected position corresponding to one of the two possible orientations, the detected orientation of the two possible orientations then being defined as the first orientation and the other as the second orientation. In steps 78 and 92 it may then check for the first orientation, and in step 98, instead of instructing the signal generation system to generate a “finished” signal, the processor may instruct the signal generation system to generate a “re-orient hand” signal. The processor may then return to step 54, now checking for the second orientation in steps 60, 78 and 92. This example method, like the method without orientation changes, may also be applied to two hands. The method and apparatus described above may also be applied to other body parts than hands, or to a person's whole body. To disinfect skin or clothes across a person's whole body, a germicidal lighting system may comprise a structure defining an opening where the opening is a pedestrian passage. For example, as shown in FIG. 5, the structure 110 may comprise a gateway 112 defining the pedestrian passage. The gateway 112 shown resembles a metal detection security checking gateway. Far-UVC lamps 114 and sensors (not shown) are arranged around the gateway on portions of the gateway facing the pedestrian passage 116. The far-UVC lamps 114 may be incorporated into the top frame 118 of the gateway, as well as into the side walls 120 as shown. The gateway may, for example, function according to the method shown in FIG. 4, with the position of the whole body replacing the position of the hand in FIG. 4. Far-UVC lamps 114 may include mirrors shaped to reflect far-UVC light out uniformly, for example in the form of parallel light as illustrated in FIG. 3.

Even where it is not practical for a pedestrian to stop while passing through a germicidal lighting system, a germicidal lighting system may still take into account the presence of a human. Sensors may detect the presence of a person within the opening and a processor may automatically start the far-UVC lamp(s) when sensing a person in a proper position within the gate, and automatically turn off the far-UVC lamp(s) when the person has left the gate. If a person remains within the opening long enough that a sufficient dose has been provided, the far-UVC lamp(s) may be turned off until another person arrives.

FIG. 6 illustrates another embodiment of a germicidal lighting system with an opening comprising a pedestrian passage. Germicidal lighting system 130 comprises a moving walkway 132 defining pedestrian passage 134 within the moving walkway. Far-UVC lamps 136 may be mounted, for example, on side walls 138 of the moving walkway supporting armrests 140. The walkway germicidal lighting system 130 may operate as described above for the gateway germicidal lighting system 110. In an alternate example, sensors (not shown) of walkway germicidal lighting system 130 can detect a position of a person or multiple people on the walkway and the germicidal lighting system 130 may turn on the far-UVC lamps 136 where people are detected. The walkway may extend a considerable distance as shown in FIG. 7, and may include many pedestrians at one time. Each far-UVC lamp 136 may be associated with a respective illumination area of the walkway and turn on as any person enters that illumination area and off when no one remains in that area. As in other embodiments, the far-UVC lamps 136 in walkway germicidal lighting system 130 may include mirrors shaped to direct the far-UVC light, for example as parallel light.

Germicidal far-UVC lighting systems operating as described here could also be incorporated into other pedestrian passages such as escalators or hallways, or into elevators. A gateway germicidal lighting system such as described above need not be a freestanding gateway; it may be, for example, incorporated into a doorframe of a doorway with or without a door.

Visible light lamps could be combined with the far-UVC lamps in any of the embodiments described here, the visible and far-UVC lamps having corresponding fields of illumination, and turned on and off together, to provide visual feedback on where and when the far-UVC disinfection is occurring.

Far-UVC light is believed to also not harm animals, and so will not harm pets that could be brought with humans through the germicidal lighting systems. The germicidal lighting systems described here may be configured to detect and disinfect animals as well as or instead of humans. They could also be configured to detect and disinfect non-living objects and/or plants as well as or instead of humans or humans and animals.

FIGS. 8-11 shows schematic drawings of an example embodiments of a far-UVC optical system 200 using a wavelength selective mirror 202. The wavelength selective mirror 202 is arranged to receive far-UVC light 203, directly or indirectly, from a far-UVC light source 204 and to disproportionately reflect the far-UVC light in at least one wavelength of the far-UVC light relative to at least another wavelength of light emitted by the far-UVC light source. For example, the wavelength selective mirror may be arranged to reflect light near a peak of an excimer lamp such as 222 nm or 207 nm, and very little or no light away from the peak. In another example, the wavelength selective mirror may be arranged to reflect light in a band of germicidal wavelengths non-harmful to humans, and little or no light away from the band. In a further example, the wavelength selective mirror may reflect a set of wavelengths excluding some but not all harmful wavelengths emitted by the far-UVC light source 204, other harmful wavelengths being excluded by a conventional transmissive filter or another wavelength selective mirror. The far-UVC light source may be, for example, an excimer lamp.

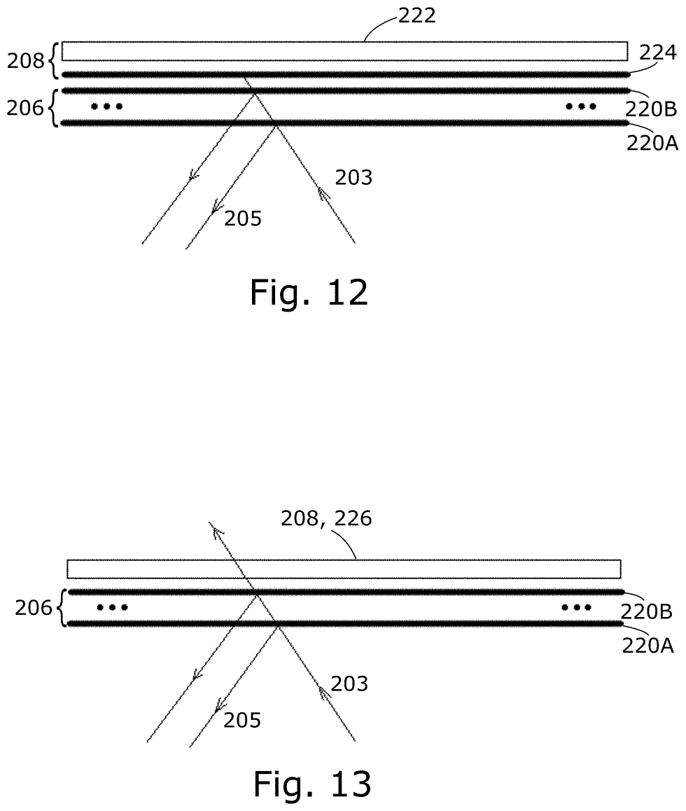

As illustrated in FIGS. 8-11, the wavelength selective mirror 202 can include a wavelength selective coating 206 on a base surface 208 of the wavelength selective mirror. In one example, the wavelength selective coating 206 may comprise a chemical that absorbs the at least another wavelength of light emitted by the far-UVC light source 204. In this case, the base surface 208 may be a reflective surface. In another example, the wavelength selective mirror may comprise one or more dielectric layers of thickness and refractive index selected to reflect the at least one wavelength, for example arranged as a Bragg mirror. This could be combined with the absorptive chemical, either in the form of a separate absorptive layer, or by integration of the absorptive chemical into the dielectric layers. In this case, the base surface 208 may be a non-reflective surface. Further examples of wavelength selective mirrors using dielectric layers are shown in FIGS. 12 and 13.

A further mirror 210 may be placed to reflect light from the far-UVC light source 204 to the wavelength selective mirror 202. This further mirror helps to direct light that would otherwise be lost into the optical system, improving efficiency. In addition, the further mirror 210 also serves to block light from avoiding the wavelength selective mirror 202 and potentially causing harmful light to escape from the far-UVC optical system 200. This blocking function could also be carried out by a non-reflective element, but a mirror is preferred for the sake of efficiency.

FIG. 8 shows a parabolic wavelength selective mirror in which the light source 204 is positioned to avoid very large changes of incidence angle. FIG. 9 shows another parabolic wavelength selective mirror in which the light source 204 is positioned closer to the wavelength selective mirror 202 to reduce the overall size of the optical system. The further mirror 210 is convex in FIG. 9 to spread reflected light over the wavelength selective mirror.

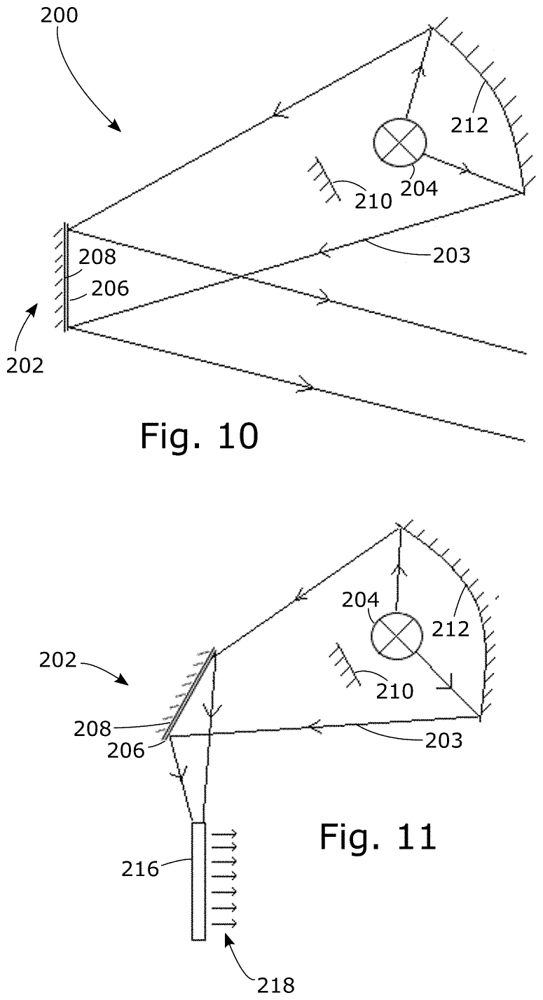

The wavelength selective mirror 202 may be one of a sequence of mirrors where each successive mirror of the sequence receives light reflected from a corresponding previous mirror of the plural mirrors arranged in the sequence. For example, as shown in FIGS. 10 and 11, a previous mirror 212 reflects light to the wavelength selective mirror 202. Additional mirrors (not shown) may be placed to receive light from the wavelength selective mirror. As shown, only one of the mirrors of the sequence is wavelength selective, but the sequence could include plural wavelength selective mirrors, for example each excluding a different set of wavelengths. The further mirror 210, or blocking member, may reflect light from the far-UVC light source 204 to the first mirror of the sequence of mirrors, regardless of whether the first mirror is the wavelength selective mirror.

Light emitted from the far-UVC optical system 200 may be converging, diverging, parallel, or diffuse. The light may reach an output of the far-UVC optical system directly from the wavelength selective mirror or another mirror, or from another element, such as a light diffusion board 216 as shown in FIG. 11. The light 218 emitted from the light diffusion board is shown as if it were parallel, but would be diffuse light.

The wavelength selective mirror 202, as well as any other mirrors, may be concave, flat or convex. A flat wavelength selective mirror, as shown in FIGS. 10-11, may be easier to manufacture; other mirrors may be shaped to obtain the desired pattern of light. Where dielectric layers are used, the wavelength response of the mirror may depend on the angle of incidence of the light on the mirror. Acceptable consistency of reflected and unreflected wavelength ranges may be obtained by avoiding large variations in incidence angle either by directing approximately parallel light to a flat mirror, as shown in FIG. 10, or by directing diverging light to a concave mirror, as shown in FIG. 8, or by directing converging light to a convex mirror (not shown). Alternatively, angle of incidence variations may be compensated by manufacturing the wavelength selective mirror to have variations in the thickness or composition of the dielectric layers in different parts of the mirror based on the expected angle of incidence.

FIGS. 12 and 13 are schematic cross sections of two example wavelength selective mirrors 202 comprising a wavelength selective coating 206 over a base layer 208. In these examples, the wavelength selective coating 206 comprises plural dielectric layers pf which two layers 220A, 220B are shown. The dielectric layers have different refractive indices from each other, for example alternating refractive indices in successive layers, and are arranged to have thicknesses and refractive indices to disproportionately reflect light including at least one wavelength of the far-UVC light from a far-UVC light source, relative to at least another wavelength of light emitted by the far-UVC light source, at an expected angle of incidence of the far-UVC light 203, e.g. due to constructive and destructive interference of reflected light 205 depending on the wavelength. In FIG. 12, the base 208 comprises a structural base layer 222 and an absorptive layer 224 that absorbs light that is transmitted through the wavelength selective coating 206. In FIG. 13, the base 208 comprises a transparent structural layer 226 that allows wavelengths transmitted through the wavelength selective coating 206 to pass through the mirror. An absorptive surface (not shown) may be placed behind the mirror. In a further example, not shown, the dielectric layers 220A . . . 220B may be collectively sufficiently thick to not require a separate structural layer; the furthest dielectric layer can then be considered the base 208.

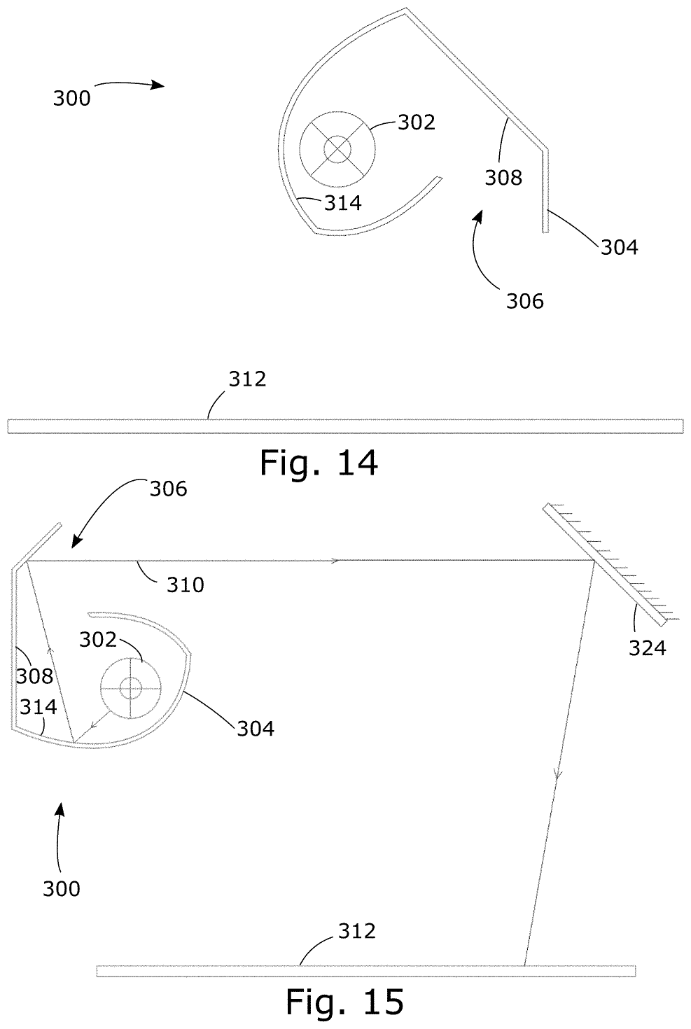

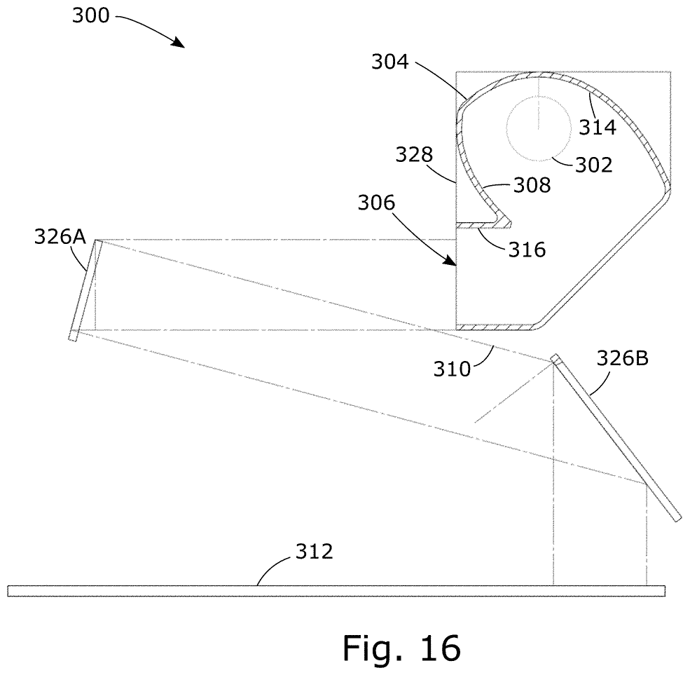

As shown in FIGS. 14-16, a far-UVC optical system 300 for a germicidal lighting system may include a far-UVC light source 302, for example an excimer lamp, and a reflective enclosure 304 around the far-UVC light source, the reflective enclosure having an opening 306 and having reflective walls 308 arranged to direct far-UVC light 310 (not shown in FIG. 14, but shown in FIGS. 15 and 16) from the far-UVC light source 302 to the opening 306. Each of FIGS. 14-16 show examples of far-UVC optical systems in cross section. The example far-UVC light sources 302 in the examples shown in FIGS. 14-16 are linear light sources with direction of linear extent perpendicular to the cross section shown. The far-UVC light 310 may be directed from the far-UVC optical system 300 to, for example, a surface 312. This may be for the purpose of irradiating the surface 312, but more typically the far-UVC optical system may be used to disinfect people for example in a hand sanitizer as shown in FIGS. 1-3 or pedestrian passage as shown in FIGS. 5-7. In such embodiments, the surface 312 may be a background behind the intended target (not shown here, but hand 16 shown in FIG. 1), or not present at all. In each of FIGS. 14-16, the reflective walls 308 include at least one concave portion 314. The term “concave” refers here and in the rest of this document to being concave in at least one cross section. The concave portion may be straight in a direction perpendicular to the cross section shown. The concave portion may focus divergent light spreading out from the light source 302 to help direct the light out of the opening 306 in a more collimated manner. The concave portion may be a continuously curved portion as shown in FIGS. 14-16 or may be formed of discrete segments (not shown) each smaller in length in the plane of the cross section shown than the width of the far-UVC lamp 302. The reflective walls 308 may form a spiral as shown in FIGS. 14-16. In FIG. 16, an extension 316 extends from an inner end of the spiral in a direction facing a path of the light 310 from the opening 306.

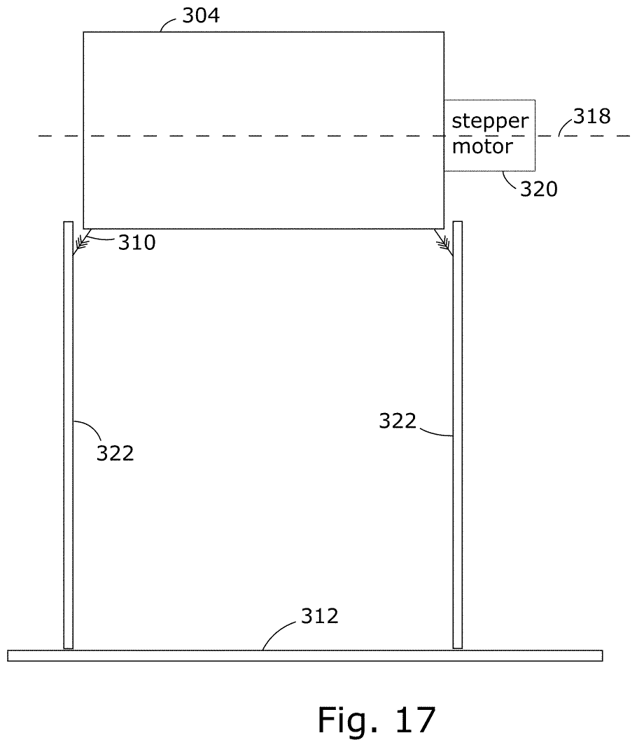

The reflective enclosure 304 may be configured to output the far-UVC light 310 from the opening 306 so that the light spreads more in one direction than another, in the embodiments shown in FIGS. 14-16 more in a direction perpendicular to the cross section shown and less in a direction within the cross section. Thus, light can reach the surface 312 as a relatively uniform strip. Features that can contribute to this differential spread of the light can include for example the far-UVC lamp source 302 being a linear light source, and the portion(s) 314 of the reflective walls 308 being concave in cross section. The reflective enclosure 304 may be mounted for rotation about an axis 318, as shown in FIG. 17. The light may spread more in a direction parallel to the axis 318 than in a direction perpendicular to the axis. This allows the rotation of the reflective enclosure 304 to move the light strip perpendicular to the length of the strip to achieve a reasonably uniform exposure of the whole surface 312 to the far-UVC light. FIG. 17 shows the reflective enclosure 304 connected to an actuator 320, here a stepper motor, to rotate the reflective enclosure 304 about the axis. The actuator rotates the reflective enclosure relative to a further structure, not shown in FIG. 17, such as the structure 12 of a germicidal lighting system 10 shown in e.g. FIG. 1. Also shown in FIG. 17 are optional side mirrors 322 arranged perpendicular to the axis to reduce losses of light to side walls of the far-UVC germicidal system in which the far-UVC optical system 300 is mounted. The stepper motor 320 may be configured to rotate the reflective enclosure 304 at a variable speed. The speed may depend on, for example, distance from the opening 306 to the illuminated portion of the surface 312, and incident angle of the light 310 to the surface 312. The variation of the speed may be used to keep the dose to the surface uniform despite the angle and distance differences to different parts of the surface. Where the target to be disinfected is a separate object in front of the surface, for example a hand, the speed may be varied depending on the known or presumed position and orientation of the hand. To further improve reliability and uniformity of illumination, multiple far-UVC optical systems 300 may be used to illuminate the hand or other target object by different angles. Where there is further mirror 324 as shown in FIG. 15, or a sequence of mirrors 326A and 326B as shown in FIG. 16, the entire set of mirrors may be included between side mirrors 322, the entire arrangement of mirrors may also be between the side mirrors 322. While only a rotational movement is shown, a translational movement would also be possible by, e.g., mounting the reflective enclosure, or any additional mirror(s), on a track for movement by an actuator.

As shown in FIG. 15, the far-UVC optical system 300 may include an additional mirror 324 arranged to receive the far-UVC light 310 after it exits the opening 306. The additional mirror 324 may be rotatable in the same manner as described above in relation to the reflective enclosure 304 being rotatable. The rotation of the additional mirror 324 may be used in combination with the reflective enclosure 304 being fixed to achieve the same objective as the reflective enclosure 304 being rotatable. Alternatively, multiple additional mirrors, rotatable or fixed, could be combined with a rotatable reflective enclosure 304 to enable the reflective enclosure to direct light from the opening 306 to different additional mirrors 324 depending on its orientation. This could be used to allow a single light source 302 to illuminate a target from different directions. As with the rotation of the reflective enclosure 304 as described above in relation to FIG. 17, rotation of the additional mirror 324 may occur at variable speed depending on the distance and angle of incidence to a target, and the reflective enclosure may output light that spreads more in a direction parallel to the axis of rotation of the additional mirror than in a direction perpendicular to the axis. Again, the far-UVC light source may be a linear light source with a direction of linear extent parallel to the axis.

As shown in FIG. 16, the far-UVC optical system 300 may include a sequence of additional mirrors, in the example shown comprising to additional mirrors 326A and 326B. The first mirror of the sequence, denoted here by 326A, may receive light from the opening 306 of the reflective enclosure 304. Each successive mirror of the sequence receives light from a corresponding previous mirror of the sequence, e.g. the mirror denoted 326B from receives light from first mirror 326A. One or more mirrors of the sequence may be rotatable. For example, the last mirror of the sequence may be rotatable, with the reflective enclosure 304 and any previous mirrors of the sequence fixed. The rotation of the last mirror of the sequence may be accomplished in the same way, and achieve the same effect, as the rotation of the additional mirror as described above or of the reflective enclosure as described in relation to FIG. 17. As with the rotation of the reflective enclosure 304 as described above in relation to FIG. 17, rotation of the last mirror 326B may occur at variable speed depending on the distance and angle of incidence to a target, and the reflective enclosure may output light that spreads more in a direction parallel to the axis of rotation of the rotating mirror than in a direction perpendicular to the axis. Again, the far-UVC light source may be a linear light source with a direction of linear extent parallel to the axis. Alternatively to the last mirror 326B being the only rotating mirror, a previous mirror 326A may also rotate, causing the light from the reflective enclosure 304 to reflect from the mirror 326A to different successive mirrors depending on the orientation of the previous mirror 326A. This could be used to allow a single light source 302 to illuminate a target from different directions.

The reflective walls 308 of the reflective enclosure 304 may also serve as structural support elements of the reflective enclosure 304, or may be inner walls supported by further structural support elements, such as outer walls 328 shown in FIG. 16. The opening 306 would also be an opening in the outer walls 328. The term “opening” in this document includes empty space or a far-UVC transparent window.

The reflective enclosure 304 may be arranged to block direct line of sight from the far-UVC light source 302 to the opening 306. In the examples shown in FIGS. 14-16, the far-UVC light source is cylindrical and line of sight from the center of the cylinder to the opening 306 is blocked. Line of sight from a portion of the circumference of the cylinder to the opening 306 is not blocked in these examples. The reflective enclosure could alternatively block line of sight from the whole of the far-UVC light source. The blocking of line of sight may be useful because, in the arrangements shown, most light is directed out of the reflective enclosure in a direction different from the line of sight from the far-UVC light source. Where most light is directed out of the reflective enclosure in the same direction as direct line of sight, for example from a parabolic mirror, and where the reflective enclosure is not relied on for wavelength selective reflection, no blocking of line of sight is needed.

In any of the embodiments including mirrors, one or more of the mirrors may be wavelength selective mirrors as described above. For example, the reflective walls 308 may be wavelength selective mirrors over all or part of the reflective walls 308, and/or the additional mirror 324 may be wavelength selective, and/or one or more mirrors of the sequence of mirrors 326A, 326B may be wavelength selective. In a preferred embodiment, at least one mirror that the light will encounter between the light source and the target is wavelength selective. The at least one wavelength selective mirror may be made wavelength selective as described above, e.g. in relation to FIGS. 8-13.

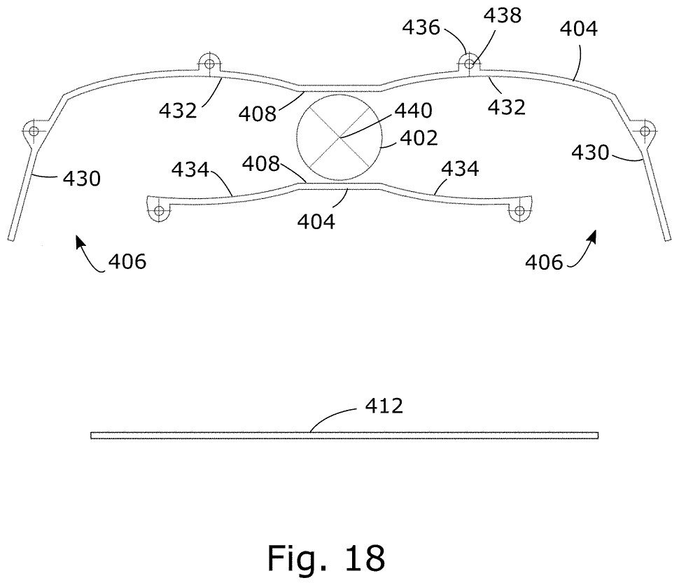

FIG. 18 shows a linear reflective enclosure 404. The linear arrangement provides direct lines of sight from the far-UVC light source 402 to end mirrors 430 which are at an intermediate position in the optical system, intermediate here meaning that they are not the ultimate target to be illuminated. Here, the end mirrors 430 direct the light through openings 406 to surface 412 or to an illuminated object that may be present above surface 412.

The reflective walls 408 of the linear reflective enclosure 404 shown in FIG. 18 include concave portions arranged around the direct lines of sight to direct generally to end mirrors 430 light emitted from the far-UVC light source 402 in directions other than the direct line of sight from the far-UVC light source 402 to end mirrors 430. These concave portions are here upper guiding mirrors 432 and lower guiding mirrors 434 shaped to direct light from the far-UVC light. The end mirrors 430 reflect the light from the far-UVC light 402, directly via the lines of sight or via the concave portions, to openings 406. These are shaped to form portions of ovals in the cross section shown. Various shapes can be used. For example, the concave portions may be shaped to form portions of one or more conic sections. Concave portions shaped as an ellipse with the far-UVC light source 402 at one focal point and the intermediate position at the other focal point of the ellipse would focus light from the far-UVC light 402 to the intermediate position. In other embodiments, the light may not be focused directly to the intermediate position, but rather, e.g. generally through the intermediate position. For example, portions shaped as a parabola could collimate light in the direction of the intermediate position. The concave portions 432 and 434 may be shaped as conic sections with or without important elements such as the light source 402 being at the focal points.

As discussed above, the term “concave” need not refer strictly to continuously curved mirrors; it may also refer to mirrors formed of discrete segments (not shown) in a concave arrangement, for example each segment smaller in length in the plane of the cross section shown than a width of the far-UVC lamp 302, or each smaller in length in the plane of the cross section than a corresponding end mirror 430 in the same plane. Larger flat mirrors may also be used in an overall concave shape. In alternative embodiments, non-concave mirrors, for example continuous flat mirrors, would also be possible. Where portions of the reflective walls 408 form, e.g. conic sections, different portions may form different conic sections. This includes the portions at different sides of the far-UVC light and portions above and below the far-UVC light.

The reflective walls 408 may be shaped so that all or substantially all of the light emitted from the far-UVC light source 402 and ultimately received at the surface 412 or other illuminated object reflects from end mirror(s) 430, either directly or after an initial reflection from another of the reflective walls 408. This enables the ultimately received light to be wavelength selective if the end mirror(s) 430 are wavelength selective, even if other mirrors are not wavelength selective. Other portions of the reflective walls 408 may also be wavelength selective if desired, or other parts of an optical chain may be wavelength selective.

The embodiment shown in FIG. 18 has direct lines of sight from the far-UVC light 402 to intermediate positions in two directions each corresponding to a different end mirror 430, and thus corresponding to a different opening 406. These lines of sight are in this embodiment directly opposite to one another, so that they overall form a straight line. In other embodiments, they could be other than directly opposite. The arrangement with them directly opposite lends itself to a relatively flat arrangement of the reflective enclosure 404, which may be convenient, for example, to attach the reflective enclosure to a ceiling of a cavity such as cavity 28 in FIG. 3.

In other embodiments, a linear reflective enclosure 404 may have only a single opening 406.

In the embodiment shown in FIG. 18, the intermediate position in the optical system which is in direct line of sight of the light source 402 is an end mirror 430 which is part of the reflective enclosure 404. In other embodiments, the intermediate position may be outside the reflective enclosure 404, the opening being in direct line of sight of the center of the light source 402. A mirror external to the reflective enclosure, as for example disclosed in FIGS. 15-16, may be at the intermediate position. For example, such a mirror may be a rotatable or otherwise movable mirror as described above. external to the reflective enclosure 404, as for example disclosed in relation to FIGS. 15-17. Further, a sequence of mirrors may be present. An external mirror or sequence of mirrors may also be present to receive light from the opening 406 even when there is an end mirror 430. Where there are multiple openings 430, a similar arrangement could exist in relation to each opening 430, or each opening 430 could have a different arrangement. The reflective enclosure 404 could also be rotatable or otherwise movable.

In the embodiment shown in FIG. 18, the far-UVC light source 402 is a linear light source, extending in a direction perpendicular to the cross sectional plane shown, so that, in this example, cross sections in planes parallel to the cross sectional plane shown would be substantially similar. In the embodiment shown in FIG. 18, the mirrors shown extend perpendicular to the section plane, the light source 402 having a corresponding direction of linear extent perpendicular to the section plane. Tabs 436 with holes 438 may accommodate reinforcement bars supporting the reflective walls 408. The reinforcement bars may extend between side walls (not shown). The side walls may include side mirrors (not shown) functioning similarly to side mirrors 322. Such side mirrors may also be included in any of the other reflective enclosures 304 disclosed in this document.

In the embodiment shown, the far-UVC light source 402 has a central axis 440. The central axis 440 has no line of sight to the openings 406 of this linear reflective enclosure 404; the same applies to other reflective enclosures 304 shown in this document. However, in an alternative embodiment, the end mirrors 430 may be replaced by separate mirrors not part of the reflective enclosure 404. These separate mirrors could be, for example, rotatable, as disclosed in relation to the additional mirrors shown in FIGS. 15 and 16. With the end mirrors 430 replaced by separate mirrors, the central axis 440 of the far-UVC light source 402 would have a direct line of sight through the openings in the linear reflective enclosure 404 to the separate mirrors. Additional mirrors could also be added, to embodiments with or without such separate mirrors replacing the end mirrors 430, and the linear reflective enclosure 404 could also be rotatable if desired.

One or more of the mirrors may be wavelength selective. For example, as mentioned above the end mirrors 430 may be wavelength selective. Where there are separate or additional mirrors, one or more mirrors of a chain of mirrors that light from the light source 402 reflects off of before reaching surface 412 may be wavelength selective.

In the claims, the word “comprising” is used in its inclusive sense and does not exclude other elements being present. The indefinite articles “a” and “an” before a claim feature do not exclude more than one of the features being present. Each one of the individual feature described here may be used in one or more embodiments and is not, by virtue only of being described here, to be construed as essential to all embodiments as defined by the claims.

1. A far-UVC optical system for a germicidal lighting system, the far-UVC optical system comprising:
a far-UVC light source; and
a wavelength selective mirror arranged to receive far-UVC light from the far-UVC light source and to disproportionately reflect the far-UVC light in at least one wavelength of the far-UVC light relative to at least another wavelength of light emitted by the far-UVC light source.

2. The far-UVC optical system of claim 1 in which the wavelength selective mirror comprises a wavelength selective coating over a base surface of the wavelength selective mirror.

3. The far-UVC optical system of claim 2 in which the wavelength selective coating comprises a chemical that absorbs the at least another wavelength and does not substantially absorb the at least one wavelength.

4-17. (canceled)

18. A germicidal lighting system for disinfecting skin or clothing of a human, the germicidal lighting system comprising:
a structure defining an opening for accommodating the human or a body part of the human;
one or more sensors arranged on the structure to generate sensor information indicative of the presence of the human or body part within the opening;
one or more far-UVC lamps arranged on the structure to emit far-UVC light to disinfect the human or human body part when the human or human body part is positioned within the opening, and
a processor connected to the sensors and to the far-UVC lamps and configured to analyze the sensor information to determine that the human or body part is present within the opening, and to activate the far-UVC lamps based on the determination that the human or body part is present within the opening.

19. The germicidal lighting system of claim 18 in which light emitted by the one or more far-UVC lamps has a wavelength within the range of 207 to 222 nm.

20-21. (canceled)

22. The germicidal lighting system of claim 18 in which the opening is a receptacle arranged to receive a hand or hands.

23. The germicidal lighting system of claim 22 in which the hand or hands are two hands, and the processor is configured to activate the one or more far-UVC lamps based on the determination that both hands are present simultaneously.

24. The germicidal lighting system of claim 22 further comprising a signal generation system, and in which the processor is configured to cause the signal generation system to generate a finishing signal based on the far-UVC light having been active for an amount of time sufficient to disinfect the hand or hands.

25. The germicidal lighting system of claim 24 in which the processor is configured to determine from the sensor information whether the hand or hands are remaining in the opening, and to cause the signal generation system to send the finishing signal based on the hand or hands having been within the opening for the amount of time.

26-38. (canceled)

39. A far-UVC optical system for a germicidal lighting system, the far-UVC optical system comprising:
a far-UVC light source; and
a reflective enclosure around the far-UVC light source, the reflective enclosure having an opening and having reflective walls arranged to direct far-UVC light from the far-UVC light source to the opening.

40. The far-UVC optical system of claim 39 in which the reflective walls comprise at least one concave portion.

41. The far-UVC optical system of claim 39 in which the opening is not in direct line of sight from at least a center of the far-UVC light source.

42. The far-UVC optical system of claim 39 in which the reflective walls are arranged as a spiral.

43. The far-UVC optical system of claim 40 in which the reflective walls include concave portions arranged around a direct line of sight from the far-UVC lamp to an intermediate position in the optical system to reflect generally to the intermediate position light emitted from the far-UVC lamp in directions other than the direct line of sight from the far-UVC lamp to the intermediate position.

44. The far-UVC optical system of claim 43 in which the far-UVC light source is a linear light source, the concave portions shaped in cross section to form portions of one or more ovals in a plane perpendicular to the light source.

45. (canceled)

46. The far-UVC optical system of claim 43 in which the reflective walls also include an end mirror at the intermediate position in the optical system, the end mirror arranged to direct to the opening the far-UVC light from the concave portions of the reflective walls and from the direct line of sight from the far-UVC lamp.

47. The far-UVC optical system of claim 46 further comprising a second end mirror in a second direct line of sight from the far-UVC lamp and arranged to direct the far-UVC light through a second opening, the second end mirror located at a second intermediate position of the optical system and second concave portions being arranged around the second direct line of sight from the far-UVC lamp to guide generally to the second intermediate position light emitted from the far-UVC lamp in directions other than the second direct line of sight from the far-UVC lamp to the second intermediate position.

48. The far-UVC optical system of claim 47 in which the second end mirror is arranged opposite to the end mirror.

49. The far-UVC optical system of claim 39 in which the reflective walls comprise a wavelength selective mirror arranged to receive the far-UVC light from the far-UVC light source and to disproportionately reflect the far-UVC light in at least one wavelength of the far-UVC light relative to at least another wavelength of light emitted by the far-UVC light source.

50-56. (canceled)

57. The far-UVC optical system of claim 39 further comprising an additional mirror arranged to receive the far-UVC light after it exits the opening.

58-72. (canceled)
